(19) United States
(12) Patent Application Publication
(10) Pub. No.: US 2024/0383836 A1
(43) Pub. Date: Nov. 21, 2024

(54) SYNTHESIS OF MULTI-RING DISALICYLATE LINKERS

(71) Applicant: EXXONMOBIL TECHNOLOGY AND ENGINEERING COMPANY, Spring, TX (US)

(72) Inventors: Matthew T KAPELEWSKI, Lambertville, NJ (US); Simon C. WESTON, Annandale, NJ (US); Catherine A. FALER, Houston, TX (US)

(21) Appl. No.: 18/785,629

(22) Filed: Jul. 26, 2024

Related U.S. Application Data
(63) Continuation of application No. PCT/US2023/061257, filed on Jan. 25, 2023.
(60) Provisional application No. 63/304,287, filed on Jan. 28, 2022.

(51) Int. Cl. C07C 51/295

(52) U.S. Cl. CPC C07C 51/295

(57) ABSTRACT

Systems and methods are provided for synthesizing multi-ring disalicylate linkers. The systems and methods can allow for synthesis of disalicylate linkers while using a reduced or minimized amount of solvent (such as down to potentially having no separate solvent) in the reaction environment. The synthesis can be performed by starting with a compound such as 4,4′-biphenol as a starting reagent. The 4,4′-biphenol (and/or other alcohol-substituted biphenyl compound) can then be exposed in a reaction environment to pressurized CO₂ in the presence of a base. The temperature and pressure in the reaction environment can be increased to achieve either supercritical conditions for the CO₂ (based on a phase diagram for neat CO₂) and/or sub-critical conditions that are substantially similar to supercritical conditions. This can allow for conversion of the 4,4′-biphenol (or other alcohol-substituted biphenyl compound) into a multi-ring disalicylate linker.

### CROSS-REFERENCE TO RELATED APPLICATIONS

This application is a Continuation of PCT/US2023/061257, filed Jan. 25, 2023, and titled “Synthesis of Multi-Ring Disalicylate Linkers”, which claims the benefit of priority to U.S. Provisional Patent Application No. 63/304,287 filed Jan. 28, 2022, and titled “Synthesis of Multi-Ring Disalicylate Linkers”, both of which are incorporated herein by reference in their entirety.

### FIELD

Methods are provided for synthesizing disalicylate compounds, such as disalicylate compounds useful as linkers for formation of metal organic framework materials.

### BACKGROUND

Metal organic framework materials are compounds that include metal ions that are coordinated by bi-dentate linkers (or other multi-dentate linkers) to form crystalline structures. Depending on the nature of the metals and the linkers, metal organic frameworks can have a variety of properties that are potentially of commercial interest.

One group of metal organic framework materials that are of interest for use in separations and/or catalytic applications are metal organic framework materials that include a multi-ring disalicylate linker. MOF-274 is an example of a metal organic framework material that includes a multi-ring disalicylate linker. Metal organic framework materials based on multi-ring disalicylate linkers can potentially be used for selective adsorption of CO₂ and/or other components from fluid streams.

One of the difficulties in applying materials such as MOF-274 in commercial applications is that multi-ring disalicylate linkers are relatively expensive to make using existing methods. Some conventional methods for synthesis of multi-ring disalicylate linkers correspond to methods that are difficult to scale up for production of commercial volumes of linker. Additionally or alternately, some synthesis methods can require high cost starting materials as reagents. What is needed is an improved method for synthesis of disalicylate linkers that can be readily scaled up for synthesis of larger volumes of linker while also reducing or minimizing costs associated with the synthesis.

U.S. Patent Application Publication 2021/0230092 describes a method for forming 4,4′-dihydroxy-[1,1′-biphenyl-3,3′-dicarboxylic acid] based on a reaction of 4,4-biphenol in an amide solvent (such as dimethylformamide) in the presence of a base. The reaction is described as being beneficial by providing a synthesis route that can be performed at lower pressures and lower temperatures than conventional synthesis methods.

### SUMMARY

In various aspects, a method of making a multi-ring disalicylate compound is provided. The method includes forming a reagent mixture. In some aspects, the reagent mixture can consist essentially of a multi-ring aromatic alcohol and a base in a reactor volume. In other aspects, the reagent mixture can include a multi-ring aromatic alcohol, a base, and a solvent in a reactor volume, with the solvent containing 50 vol % or more of dioxane. The method further includes adding CO₂ to the reactor volume. The method further includes heating the reactor volume to a process temperature of 150° C. or more, the reactor volume having a total pressure of 1.0 MPa-a or more at the process temperature. Additionally, the method includes maintaining the reactor volume at the process temperature for 0.5 hours or more to form a product mixture including a multi-ring disalicylate product.

### BRIEF DESCRIPTION OF THE DRAWINGS

FIG. 1 shows H-NMR characterization of the reaction products from forming a multi-ring disalicylate product according to the methods described herein.

FIG. 2 shows ¹³C-NMR characterization of the reaction products from forming a multi-ring disalicylate product according to the methods described herein.

FIG. 3 shows another example of H-NMR characterization of the reaction products from forming a multi-ring disalicylate product according to the methods described herein.

### DETAILED DESCRIPTION

All numerical values within the detailed description and the claims herein are modified by “about” or “approximately” the indicated value, and take into account experimental error and variations that would be expected by a person having ordinary skill in the art.

### Overview

In various aspects, systems and methods are provided for synthesizing multi-ring disalicylate linkers. The systems and methods can allow for synthesis of disalicylate linkers while using a reduced or minimized amount of solvent (such as down to potentially having no separate solvent) in the reaction environment. The synthesis can be performed by starting with a compound such as 4,4′-biphenol as a starting reagent. The 4,4′-biphenol (and/or other alcohol-substituted biphenyl compound) can then be exposed in a reaction environment to pressurized CO₂ in the presence of a base. In some aspects, the base can correspond to a solid base, such as KHCO₃, K₂CO₃, NaOH, or another convenient choice. The temperature and pressure in the reaction environment can be increased to achieve either supercritical conditions for the CO₂ (based on a phase diagram for neat CO₂) and/or sub-critical conditions that are substantially similar to supercritical conditions. This can allow for conversion of the 4,4′-biphenol (or other alcohol-substituted biphenyl compound) into a multi-ring disalicylate linker.

Traditionally, formation of multi-ring disalicylate linkers has been performed in a solvent environment. Due to relatively low solubility in water of reagents for forming multi-ring disalicylate linkers, the traditional solvents correspond to solvents such as trichlorobenzene or dimethylformamide. Such solvent-based synthesis methods are effective for making multi-ring disalicylate linkers, but present several challenges with regard to scaling up production of metal organic framework materials for commercial scale production. First, such solvent-based synthesis methods can often present difficulties when attempting to change the synthesis conditions from a laboratory scale beaker to a commercial scale production tank. Second, due to the hazardous nature of solvents such as trichlorobenzene or dimethylformamide, it is desirable to avoid having large reaction vessels that contain substantial volumes of such substances.

In contrast to such traditional methods, it has been discovered that multi-ring disalicylate compounds can be formed by exposing a multi-ring aromatic alcohol compound to CO₂ in the presence of a base and under temperature and pressure conditions that are supercritical or approach supercritical. The temperatures and pressures involved in achieving a supercritical state for CO₂ (according to the phase diagram for neat CO₂) are temperatures and pressures that are well understood from a reactor design perspective. By using such elevated temperatures and pressures, the need for solvents can be substantially or even entirely avoided. While a solvent can be included in the reaction environment, because the solvent is not required, the volumes of solvent included in the reaction environment can be maintained at a relatively low volume. Additionally, since a solvent is not required, the “solvent” included in the reaction environment can optionally correspond to a solvent that provides only limited solubility for the reagents used to make the multi-ring disalicylate linker.

It is noted that the reaction conditions for the synthesis described herein are similar to the reaction condition for the Kolbe-Schmitt reaction that can be used to make, for example, salicylic acid. However, by using a multi-ring aromatic alcohol compound as the multi-ring reagent, a variety of process advantages can be achieved. First, the reaction can be performed at sub-supercritical reaction pressures relative to the phase diagram for pure CO₂. This can be beneficial for commercial scale up, as the structural requirements for the pressurized vessel for performing the reaction can be reduced or minimized.

Second, depending on the target compound, the synthesis conditions for forming multi-ring disalicylate compounds can provide an unexpected advantage with regard to product purity relative to a conventional Kolbe-Schmitt reaction. In particular, depending on the target disalicylate product, forming a multi-ring disalicylate can avoid stereochemistry uncertainties that can be associated with a conventional Kolbe-Schmitt process. When forming salicylic acid from sodium phenoxide using a conventional Kolbe-Schmitt reaction, CO₂ addition can potentially occur at both the ortho-and para-positions on the aromatic ring. By contrast, depending on the target disalicylate product, a higher degree of control over the reaction products can be achieved. For example, when 4,4-biphenol is used as the multi-ring reagent, the para-position is occupied by the biphenyl linkage, and therefore is not available for reaction with a CO₂. Due to the free rotation around this biphenyl linkage, reaction at either ortho-position results in the same product. This provides the unexpected benefit of being able to use a Kolbe-Schmitt style process while producing a reaction product that is substantially free of isomers.

In addition to benefits relative to a Kolbe-Schmitt style process, synthesis of multi-ring disalicylate compounds as described herein can share some of the benefits of a Kolbe-Schmitt style process. For example, Kolbe-Schmitt type reactions are already used industrially for production of salicylic acid (as a precursor for production of aspirin). Thus, in contrast to laboratory scale methods for production of multi-ring disalicylates, it is believed that the synthesis methods described herein can be readily scaled for production of commercial volumes of multi-ring disalicylate compounds.

Still another benefit of using high pressure, high temperature CO₂ as the reaction environment is that such an environment can simplify recovery of the disalicylate product compound. Reagents such as 4,4′-biphenol have a relatively low solubility in water or alcohol. By contrast, multi-ring disalicylate compounds typically are soluble in water, and often have solubility in various types of alcohol. As a result, after performing a synthesis reaction to form a multi-ring disalicylate, the product compound (disalicylate) can be separated from unreacted multi-ring reagent (such as 4,4-biphenol) by adding water to the product. The unreacted multi-ring reagent can be separated from the resulting mixture by filtration. The water can then be acidified to precipitate out the target disalicylate product.

It is noted that the product compounds from this synthesis are described as disalicylates. It is understood that with a suitable starting reagent that has three or more alcohol groups bonded to aromatic rings, compounds with more than two salicylate groups could also be formed. In this discussion, the product compounds are described as disalicylates for convenience in describing the synthesis method.

### Definitions

In this discussion, a multi-ring compound that includes two or more hydroxyls attached in aromatic rings is defined as a multi-ring aromatic alcohol, independent of other substituents that may also be present within the compound. In some aspects, carbon atoms in at least two different aromatic rings in a multi-ring aromatic alcohol can correspond to carbon atoms that are attached to a hydroxyl group. Under this definition, rings within a fused ring structure are counted as separate rings, so naphthalene (C₁₀H₈) is defined as a compound that includes two aromatic rings.

In various aspects, the aromatic rings in a multi-ring aromatic alcohol can include only carbon atoms, so that “heteroatoms” such as nitrogen or oxygen are not present in the aromatic rings of the multi-ring aromatic alcohol. Without being bound by any particular theory, it is believed that the presence of such heteroatoms can disrupt the reaction mechanism.

The term “multi-ring” is defined herein to refer to compounds that include two or more ring structures (i.e., cyclic structures). The rings can correspond to fused rings, such as a naphthalene-type structure, rings bonded together without sharing an atom, such as a biphenyl linkage, or rings separated by one or more atoms, such as rings separated by a methyl linkage. This is in contrast to a single-ring compound. A multi-ring compound can include multiple aromatic rings, multiple non-aromatic rings (such as saturated rings and/or rings including an insufficient number of double bonds to provide aromaticity), or a combination thereof.

In this discussion, a reactor is defined as any vessel, container, pipe, or other structure that can be used to provide a pressurized, high temperature reaction environment for performing the reaction(s) described herein.

### Synthesis Method and Product Recovery

In various aspects, a multi-ring disalicylate compound can be formed by exposing reagents to CO₂ under high temperature, high pressure reaction conditions. The reaction environment for forming a multi-ring disalicylate compound can include a) a multi-ring alcohol reagent that includes two or more hydroxyl groups bonded to carbons in an aromatic ring, b) a base, and c) CO₂.

In some aspects, the reagents in the reaction environment can consist essentially of the multi-ring alcohol, the base, and CO₂. In such aspects wherein the reagents in the reaction environment consist essentially of the multi-ring alcohol, the base, and CO₂, other components can be in the reaction environment, such as inert gases (e.g., N²). Additionally, in such aspects wherein the reagents in the reaction environment consist essentially of the multi-ring alcohol, the base, and CO₂, water may be present due to waters of hydration associated with the reagents, although in some aspects it may be preferable to dry the reagents to remove waters of hydration prior to synthesis. Any water present as waters of hydration can combine with water that is evolved in-situ during the reaction. Optionally, all of the reagents can be in solid form prior to the start of the synthesis procedure.

In other aspects, a solvent can also be present. If a solvent is present, the solvent can preferably correspond to 50 vol % or more dioxane. It is noted that traditional synthesis solvents for disalicylates such as trichlorobenzene or dimethylformamide could also be included, but such inclusion would tend to reduce some of the benefits of the synthesis method. In particular, inclusion of such solvents can tend to increase the restrictions and/or special procedures required for performing the reaction.

The multi-ring alcohol reagent can correspond to a multi-ring compound that includes at least two hydroxyl (—OH) groups attached to carbons in aromatic rings within the compound. The reagent can be selected so that the relative location of the alcohol groups in the reagent matches the location of the hydroxyl group portions of the salicylate groups in the desired or target compounds. At least one “ortho” location on the aromatic ring relative to each hydroxyl group also needs to be available to allow for addition of a CO₂ to form the carboxylate group. In some aspects, some benefit may be provided by having both “ortho” locations available, so as to reduce or minimize any negative effects on yield due to having a substituent at a location that is “meta” relative to where the carboxylate group will be added. It is noted that formation of a disalicylate requires the presence of at least two alcohol groups in the multi-ring alcohol reagent compound.

One example of a multi-ring alcohol reagent is 4,4′-biphenol. By adding a carboxylate group at an ortho position relative to each alcohol group, the compound 4,4′-dihydroxy-1,1′-biphenyl-3,3′-dicarboxylic acid can be formed. This compound can be referred to as H₄DOBPDC. It is noted that due to the free rotation around the biphenyl bond and the lack of a chiral center, addition of a carboxylate at either ortho position results in production of the same compound.

Another reagent can be a base. Examples of suitable bases include, but are not limited to, alkali carbonates, alkali bicarbonates, and alkali hydroxides. Examples of bases include, but are not limited to, KOH, KHCO₃, K₂CO₃, NaOH, NaHCO₃, Na₂CO₃, and mixtures thereof. It is noted that the base plays a stoichiometric role within the reaction environment, resulting in formation of water as a by-product of the reaction that adds the carboxylate group to a ring. Additionally, CO₂ serves as both reagent and reaction medium.

To start synthesis of a multi-ring disalicylate compound, a multi-ring alcohol reagent, a base, and CO₂ can be added to a reactor. The reactor can then be heated and pressurized. The reactor can be heated and pressurized in any convenient order. For example, the reactor can initially be pressurized to a first pressure value by addition of gas phase CO₂ at a temperature near 25° C. After adding the desired amount of CO₂, the reactor can then be heated. This will result in further increases in pressure due to the CO₂ either being in the gas phase or being present as a supercritical fluid. The pressurization and heating can be performed in any convenient manner to achieve a target set of conditions for performing the reaction to form the multi-ring disalicylate compound.

In various aspects, the reaction can be performed by maintaining the reaction environment at a temperature of 150° C. or higher, or 200° C. or higher, or 250° C. or higher, such as up to 500° C. or possibly still higher. The temperature can be maintained within such a target range for a reaction time. With regard to pressure, in various aspects the total pressure within the reaction environment can substantially correspond to the pressure of CO₂ in the reaction environment. In various aspects, the total pressure in the reaction environment can be maintained at a pressure of 1.0 MPa-a or more, or 3.5 MPa-a or more, or 6.0 MPa-a or more, or 7.38 MPa-a or more, such as up to 20 Mpa-a or possibly still higher. In some aspects, the reaction environment can be maintained at total pressures below the supercritical point for pure CO₂ for a reaction time. In such aspects, the total pressure can be between 1.0 MPa-a to 7.35 MPa-a (i.e., below the supercritical pressure of 7.38 MPa-a) for a reaction time, or 3.5 MPa-a to 7.35 MPa-a, or 5.0 MPa-a to 7.35 MPa-a, or 6.0 MPa-a to 7.35 MPa-a, or 1.0 MPa-a to 7.0 MPa-a, or 1.0 MPa-a to 6.5 MPa-a, or 3.5 MPa-a to 7.0 MPa-a. In other aspects, the reaction environment can be maintained at total pressures at or above the supercritical point for CO₂ for a reaction time. In such aspects, the total pressure can be 7.38 MPa-a to 20 MPa-a, or 7.38 MPa-a to 12 MPa-a, or 7.5 MPa-a to 20 MPa-a, or 7.5 MPa-a to 12 MPa-a. It is noted that the total pressure in the reaction environment can be higher than the CO₂ pressure due to the presence of other fluids. For example, water is evolved during the reaction to form the multi-ring disalicylate product. While the molar amount of water will typically be small relative to the molar amount of CO₂ in the reaction environment, such water could nonetheless contribute to the total pressure being slightly higher than the CO₂ pressure. In aspects where a separate solvent (such as dioxane) is added to the reagent mixture, the difference between the total pressure and the CO₂ partial pressure can be larger.

The reaction environment can be maintained at a temperature and/or pressure within the target ranges for a reaction time of 0.5 hours to 48 hours, or 0.5 hours to 24 hours, or 0.5 hours to 12 hours, or 0.5 hours to 6.0 hours, or 4.0 hours to 8.0 hours. It is noted that longer times could also be used, but such longer reaction times can tend to reduce the throughput that can be achieved for a reactor.

The amount of multi-ring aromatic alcohol reagent and base in the reaction environment can be selected so that a molar ratio of alcohol reagent to base in the reaction environment is between 0.3 and 3.0 (i.e., between 0.3 to 1 and 3.0 to 1), or between 0.15 to 3.0, or between 0.15 to 2.0, or between 0.5 to 2.0, or between 0.8 to 1.2. The amount of CO₂ can be any convenient amount so that a substantial molar excess of CO₂ is present in the reaction environment relative to the amount of multi-ring aromatic alcohol reagent, such as having a molar amount of CO₂ that is at least 5.0 times the molar amount of the multi-ring aromatic alcohol reagent. It is noted that if CO₂ is used to at least partially pressurize the reaction environment, a substantial molar excess of CO₂ will typically be present.

Optionally, a solvent can also be included in the reaction environment. Dioxane is an example of such a solvent. In some aspects, the amount of solvent can be reduced or minimized, so that the weight of the solvent is comparable to or less than the combined weight of the multi-ring aromatic alcohol and the base in the reaction environment. In such aspects, the weight of the solvent can be less than 3.0 times the combined weight of the multi-ring aromatic alcohol and the base, or less than 2.0 times the combined weight, or less than 1.0 times the combined weight, or less than 0.5 times the combined weight, such as down to having substantially no solvent in the reaction environment. It is noted that the reaction process for forming the multi-ring disalicylate compound stoichiometrically creates one water for each salicylate group that is formed, so a small amount of water will be created in-situ in the reaction environment even if the multi-ring aromatic alcohol and the base are introduced into the reaction environment as dry, solid reagents.

It is noted that using water alone as a solvent is not effective for formation of disalicylate linkers under the synthesis conditions described herein. However, it has been unexpectedly discovered that dioxane can be used as a solvent. It is unexpected that dioxane can be used as a solvent when water cannot based on the dipole moments of the solvents. In particular, it is known that dimethylformamide (dipole moment 3.86 Debyes) can be used as a solvent. Water (dipole moment 1.85 Debyes) does not result in meaningful production of disalicylates when used as a solvent. However, dioxane (dipole moment 0.45 Debyes) does result in disalicylate production.

After maintaining the reaction environment in a target range for pressure and temperature for a reaction time, the reaction can be stopped. The multi-ring disalicylate product can then be recovered by any convenient method. One option can be to add water (if needed) to form a solution containing the multi-ring disalicylate product. The solubility of the multi-ring aromatic alcohol reagent is typically relatively low in water, so the resulting water mixture can be filtered, for example, to remove the solid multi-ring aromatic alcohol reagent. It is noted that any multi-ring aromatic alcohol reagent that is recovered from the products can potentially be recycled for use again as reagent. After filtering, the water mixture can be acidified to precipitate out the multi-ring disalicylate product. The solid product can then be recovered by any convenient method for separating a solid from a liquid.

### Multi-Ring Disalicylate Compound Structures

Generally, the synthesis methods described herein can be used to make disalicylate compounds. A disalicylate corresponds to a compound that includes two monohydroxybenzoate groups.

In an aspect, useful disalicylate linkers include:

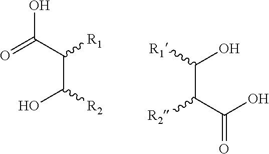

where R₁ is connected to R₁′ and R₂ is connected to R₂.″

Examples of such linkers include:

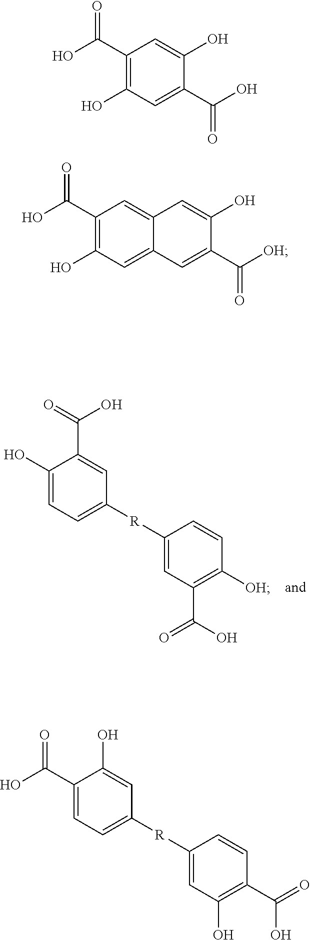

where R is any molecular fragment.

Some examples of disalicylate compounds can be have two phenyl rings joined at carbon 1,1′ (i.e., a biphenyl type linkage), with carboxylic acids on carbons 3, 3′, and alcohols on carbons 4,4′. This compound can be referred to as “H₄DOBPDC”.

Other examples of disalicylates can include para-carboxylate (“pc-linker”) such as 4,4′-dioxidobiphenyl-3,3′-dicarboxylate (DOBPDC); 4,4″-dioxido-[1,1′: 4′,1″-terphenyl]-3,3″-dicarboxylate (DOTPDC); and dioxidobiphenyl-4,4′-dicarboxylate (para-carboxylate-DOBPDC also referred to as PC-DOBPDC) as well as the following compounds:

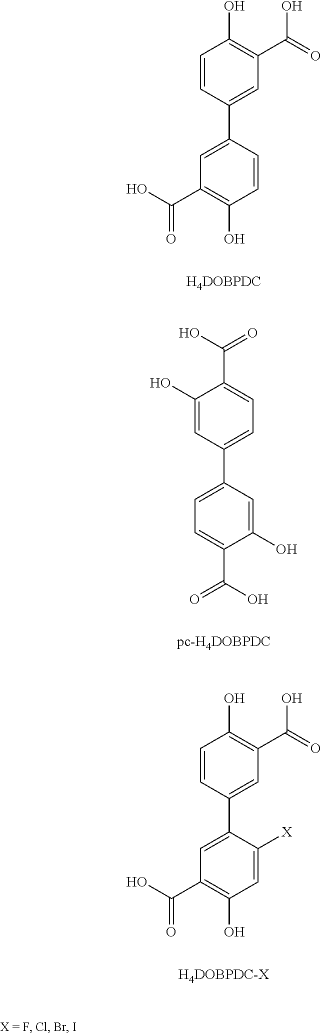

In an aspect, the disalicylate has the formula:

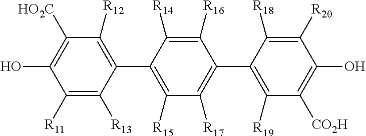

where R₁₁, R₁₂, R₁₃, R₁₄, R₁₅, R₁₆, R₁₇, R₁₈, R₁₉, and R₂₀ are each independently selected from H, halogen, hydroxyl, methyl, and halogen substituted methyl.

In an aspect, the disalicylate has the formula:

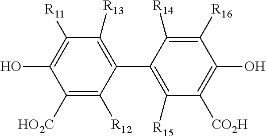

where, R₁₁, R₁₂, R₁₃, R₁₄, R₁₅, and R₁₆ are each independently selected from H, halogen, hydroxyl, methyl, and halogen substituted methyl.

In an aspect, the organic linker has the formula:

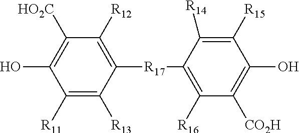

where R₁₁, R₁₂, R₁₃, R₁₄, R₁₅, and R₁₆ are each independently selected from H, halogen, hydroxyl, methyl, or halogen substituted methyl, and R₁₇ is selected from substituted or unsubstituted aryl, vinyl, alkynyl, and substituted or unsubstituted heteroaryl.

In an aspect, the organic linker has the formula:

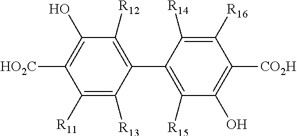

where R₁₁, R₁₂, R₁₃, R₁₄, R₁₅, and R₁₆ are each independently selected from H, halogen, hydroxyl, methyl, or halogen substituted methyl.

where R₁₁, R₁₂, R₁₃, R₁₄, R₁₅, and R₁₆ are each independently selected from H, halogen, hydroxyl, methyl, or halogen substituted methyl, and R₁₇ is selected from substituted or unsubstituted aryl, vinyl, alkynyl, and substituted or unsubstituted heteroaryl.

In an aspect, the organic linker includes multiple bridged aryl species such as molecules having two (or more) phenyl rings or two phenyl rings joined by a vinyl or alkynyl group.

It is noted that the above examples primarily correspond to structures where two or more aromatic rings are joined via a biphenyl linkage. In other aspects, a multi-ring disalicylate linker can include an aromatic core of two or more fused aromatic rings.

### EXAMPLE 1

### Synthesis of H₄DOBPDC From 4,4′-biphenol (No Added Solvent)

To prepare H₄DOBPDC (4,4′-dihydroxy-1,1′-biphenyl-3,3′-dicarboxylic acid), 10 g of 4,4′-biphenol was ground up and dried in an oven at 120° C. for roughly 3 hours. Separately, 16.13 g of a solid base (KHCO₃) was ground up and dried in an oven at 120° C. The two solids were mixed together thoroughly in a glass vessel, placed in an autoclave, sealed, and purged with N₂. The vessel was then pressurized with CO₂ to roughly 3.7 MPa-a. The vessel was then heated to 280° C., resulting in an increase in the pressure in the vessel to roughly 7.6 MPa-a. As a result, the CO₂ inside the vessel corresponded to supercritical CO₂ (based on the phase diagram for pure CO₂). The vessel was maintained at the combination of temperature and pressure for roughly 24 hours. The reactor was then cooled and depressurized.

The resulting reaction products inside the vessel corresponded a mixture of solids. Water was added to the solids to form a mixture of solids and water. The mixture of solids and water was sonicated and then stirred vigorously for at least an hour. The resulting slurry was then filtered. The solids removed by filtration substantially corresponded to unreacted 4,4′-biphenol. The filtrate was then acidified with HCl to a pH of less than 2. This resulted in precipitation of a white solid. Filtration was used to recover the white solid. The white solid substantially corresponded to substantially pure H₄DOBPDC. It is noted that re-crystallization could be performed to increase product purity.

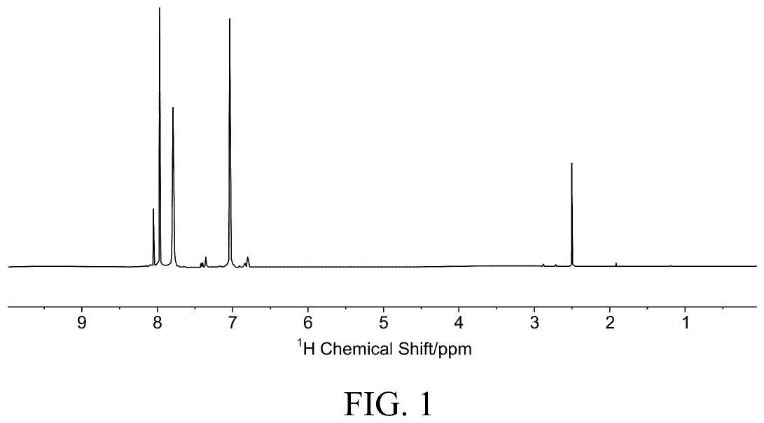

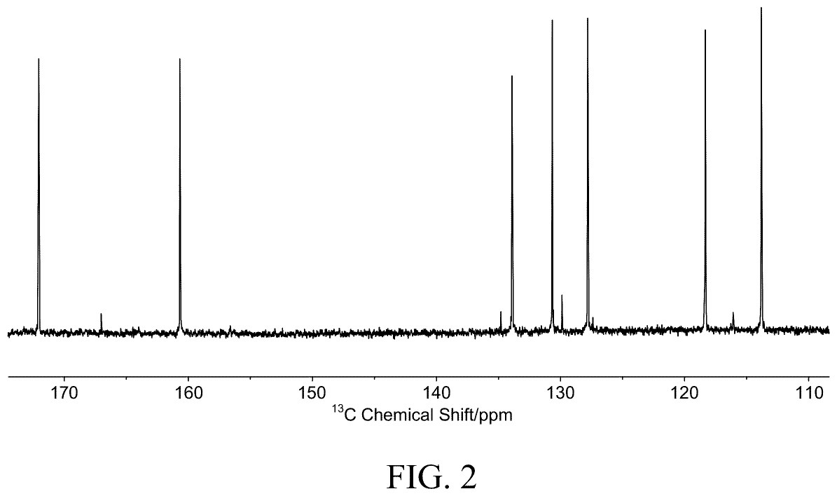

H₄DOBPDC samples formed according to the above method were characterized using H-NMR and ¹³C-NMR. FIG. 1 shows an example of an H-NMR spectrum and FIG. 2 shows an example of a ¹³C-NMR spectrum obtained for relatively pure H⁴DOBPDC samples generated by the methods described herein. The spectra shown in FIG. 1 and FIG. 2 illustrate that H₄DOBPDC was obtained.

### EXAMPLE 2

### Synthesis of H₄DOBPDC From 4,4′-biphenol (With Dioxane as Solvent)

To prepare H: DOBPDC (4,4′-dihydroxy-1,1′-biphenyl-3,3′-dicarboxylic acid), 3.0 g of 4,4′-biphenol and 6.68 g of freshly ground K₂CO₃ were mixed with stirring in 40 mL of 1,4-dioxane. The resulting solution was placed in an autoclave, sealed, and purged with N₂. The vessel was then pressurized with CO₂ to roughly 3.7 MPa-a. The vessel was then heated to 280° C. overnight. After bringing the reactor to ambient temperature and pressure, the contents were dissolved in water and then acidified using 10% HCl in water. This resulted in a precipitation of a solid, which was recovered from the solution by filtration. The solid was then dissolved in acetone. dried over MgSO. and filtered. Concentration of the filtrate gave the diacid as a pale yellow solid.

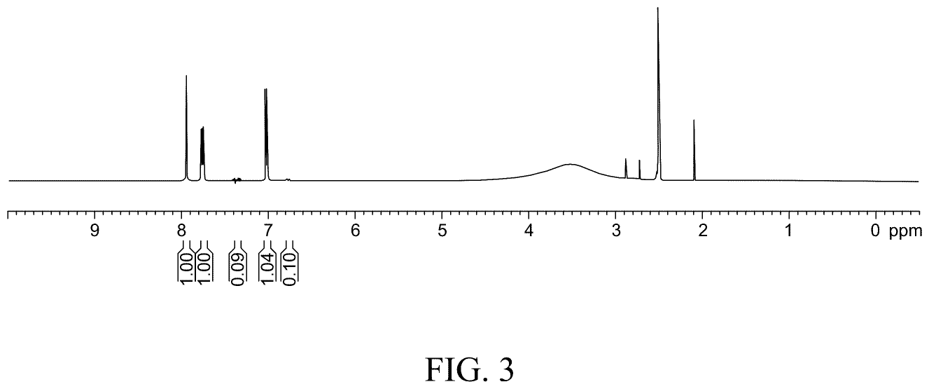

A sample of the pale yellow solid was dissolved in dimethyl sulfoxide (DMSO) to allow for characterization via H-NMR. FIG. 3 shows the resulting spectrum, which indicates that H₄DOBPDC was obtained.

It is noted that other convenient acids can be used to acidify the water to facilitate precipitation. For example, sulfuric acid can also be used. Additionally or alternately, lower concentrations of acid can be used as the reagent for acidifying a solution.

### ADDITIONAL EMBODIMENTS

Embodiment 1. A method of making a multi-ring disalicylate compound, comprising: forming a reagent mixture i) consisting essentially of a multi-ring aromatic alcohol and a base; or ii) comprising a multi-ring aromatic alcohol, a base, and a solvent in a reactor volume, the solvent comprising 50 vol % or more of dioxane; adding CO₂ to the reactor volume; heating the reactor volume to a process temperature of 150° C. or more, the reactor volume comprising a total pressure of 1.0 MPa-a or more at the process temperature; and maintaining the reactor volume at the process temperature for 0.5 hours or more to form a product mixture comprising a multi-ring disalicylate product.

Embodiment 2. The method of Embodiment 1, further comprising separating at least a portion of the multi-ring disalicylate product from the product mixture.

Embodiment 3. The method of any of the above embodiments, wherein the total

pressure at the process temperature is 1.0 MPa-a to 7.35 MPa-a.

Embodiment 4. The method of any of the above embodiments, wherein the total pressure at the process temperature is 1.0 MPa-a to 6.5 MPa-a.

Embodiment 5. The method of Embodiment 1 or 2, wherein the total pressure at the process temperature is 7.40 MPa-a to 20 MPa-a.

Embodiment 6. The method of any of the above embodiments, wherein the process temperature is 150° C. to 500° C.

Embodiment 7. The method of any of the above embodiments, wherein forming the reagent mixture comprises forming a reagent mixture having a molar ratio of multi-ring aromatic alcohol to base of 0.5 to 2.0.

Embodiment 8. The method of any of the above embodiments, wherein the multi-ring aromatic alcohol comprises 4,4′-biphenol.

Embodiment 9. The method of any of the above embodiments, wherein the base comprises an alkali carbonate, an alkali bicarbonate, an alkali hydroxide, or a combination thereof.

Embodiment 10. The method of any of the above embodiments, wherein the multi-ring

aromatic alcohol comprises at least one hydroxyl group bonded to a carbon in an aromatic ring wherein each carbon atom in an ortho position in the aromatic ring is bonded to a hydrogen.

Embodiment 11. The method of any of the above embodiments, wherein forming the reagent mixture comprises forming a reagent mixture having a molar ratio of solvent to multi-ring aromatic alcohol of 3.0 or less.

Alternative Embodiment A. A method of making a multi-ring disalicylate compound, comprising: forming a reagent mixture consisting essentially of a multi-ring aromatic alcohol and a base in a reactor volume; adding CO₂ to the reactor volume; heating the reactor volume to a process temperature of 150° C. or more, the reactor volume comprising a total pressure of 1.0 MPa-a or more at the process temperature; and maintaining the reactor volume at the process temperature for 0.5 hours or more to form a product mixture comprising a multi-ring disalicylate product.

Alternative Embodiment B. A method of making a multi-ring disalicylate compound, comprising: forming a reagent mixture comprising a multi-ring aromatic alcohol, a base, and a solvent in a reactor volume, the solvent comprising 50 vol % or more of dioxane; adding CO₂ to the reactor volume; heating the reactor volume to a process temperature of 150° C. or more, the reactor volume comprising a total pressure of 1.0 MPa-a or more at the process temperature; and maintaining the reactor volume at the process temperature for 0.5 hours or more to form a product mixture comprising a multi-ring disalicylate product.

Additional Embodiment C. The method of any of Embodiments 1 to 6 or 8 to 11, wherein forming the reagent mixture comprises forming a reagent mixture having a molar ratio of multi-ring aromatic alcohol to base of 0.15 to 2.0.

Certain features have been described using a set of numerical upper limits and a set of numerical lower limits. It should be appreciated that ranges from any lower limit to any upper limit are contemplated unless otherwise indicated. Certain lower limits, upper limits and ranges appear in one or more claims below. All numerical values take into account experimental error and variations that would be expected by a person having ordinary skill in the art.

The foregoing description of the disclosure illustrates and describes the present methodologies. Additionally, the disclosure shows and describes exemplary methods, but it is to be understood that various other combinations, modifications, and environments may be employed and the present methods are capable of changes or modifications within the scope of the concept as expressed herein, commensurate with the above teachings and/or the skill or knowledge of the relevant art.

We claim:

1. A method of making a multi-ring disalicylate compound, comprising:
forming a reagent mixture
i) consisting essentially of a multi-ring aromatic alcohol and a base; or
ii) comprising a multi-ring aromatic alcohol, a base, and a solvent in a reactor volume, the solvent comprising 50 vol % or more of dioxane;
adding CO₂ to the reactor volume;
heating the reactor volume to a process temperature of 150° C. or more, the reactor volume comprising a total pressure of 1.0 MPa-a or more at the process temperature; and
maintaining the reactor volume at the process temperature for 0.5 hours or more to form a product mixture comprising a multi-ring disalicylate product.

2. The method of claim 1, further comprising separating at least a portion of the multi-ring disalicylate product from the product mixture.

3. The method of claim 1, wherein the total pressure at the process temperature is 1.0 MPa-a to 7.35 MPa-a.

4. The method of claim 3, wherein the total pressure at the process temperature is 1.0 MPa-a to 6.5 MPa-a.

5. The method of claim 1, wherein the total pressure at the process temperature is 7.40 MPa-a to 20 MPa-a.

6. The method of claim 1, wherein the process temperature is 150° C. to 500° C.

7. The method of claim 1, wherein forming the reagent mixture comprises forming a reagent mixture having a molar ratio of multi-ring aromatic alcohol to base of 0.5 to 2.0.

8. The method of claim 1, wherein forming the reagent mixture comprises forming a reagent mixture having a molar ratio of multi-ring aromatic alcohol to base of 0.15 to 2.0.

9. The method of claim 1, wherein the multi-ring aromatic alcohol comprises 4,4′-biphenol.

10. The method of claim 1, wherein the base comprises an alkali carbonate, an alkali bicarbonate, an alkali hydroxide, or a combination thereof.

11. The method of claim 1, wherein the multi-ring aromatic alcohol comprises at least one hydroxyl group bonded to a carbon in an aromatic ring wherein each carbon atom in an ortho position in the aromatic ring is bonded to a hydrogen.

12. The method of claim 1, wherein forming the reagent mixture comprises forming a reagent mixture having a molar ratio of solvent to multi-ring aromatic alcohol of 3.0 or less.

13. The method of claim 1, wherein the reagent mixture consists essentially of a multi-ring aromatic alcohol and a base.

14. The method of claim 1, wherein the reagent mixture comprises a multi-ring aromatic alcohol, a base, and a solvent.

15. A method of making a multi-ring disalicylate compound, comprising:
forming a reagent mixture consisting essentially of a multi-ring aromatic alcohol and a base in a reactor volume;
adding CO₂ to the reactor volume;
heating the reactor volume to a process temperature of 150° C. or more, the reactor volume comprising a total pressure of 1.0 MPa-a or more at the process temperature; and
maintaining the reactor volume at the process temperature for 0.5 hours or more to form a product mixture comprising a multi-ring disalicylate product.

16. A method of making a multi-ring disalicylate compound, comprising:
forming a reagent mixture comprising a multi-ring aromatic alcohol, a base, and a solvent in a reactor volume, the solvent comprising 50 vol % or more of dioxane;
adding CO₂ to the reactor volume;
heating the reactor volume to a process temperature of 150° C. or more, the reactor volume comprising a total pressure of 1.0 MPa-a or more at the process temperature; and
maintaining the reactor volume at the process temperature for 0.5 hours or more to form a product mixture comprising a multi-ring disalicylate product.
